(19) United States
(12) Patent Application Publication
(10) Pub. No.: US 2017/0089883 A1
(43) Pub. Date: Mar. 30, 2017

(54) CELL EVALUATION DEVICE, METHOD, AND PROGRAM

(71) Applicant: FUJIFILM Corporation, Tokyo (JP)

(72) Inventor: Takashi WAKUI, Ashigarakami-gun (JP)

(73) Assignee: FUJIFILM Corporation, Tokyo (JP)

(21) Appl. No.: 15/274,067

(22) Filed: Sep. 23, 2016

(30) Foreign Application Priority Data
Sep. 29, 2015 (JP) 2015-190861

(51) Int. Cl. G01N 33/50; G01N 35/00; C12M 1/34

(52) U.S. Cl. CPC G01N 33/5005; C12M 41/46; G01N 35/00722; G01N 2035/00891

(57) ABSTRACT

Cell evaluation device includes a first evaluation unit that evaluates the similarity between cells to be evaluated and cell species, which are located closer to a non-differentiation side than the cells to be evaluated are in the process of differentiation of the cells to be evaluated, as a first similarity, a second evaluation unit that evaluates the similarity between the cells to be evaluated and cell species, which are located closer to a differentiation side than the cells to be evaluated are in the process of differentiation of the cells to be evaluated, as a second similarity, and a differentiation progress calculation unit that calculates the degree of progress of differentiation of the cells to be evaluated based on the first similarity and the second similarity.

### CROSS-REFERENCE TO RELATED APPLICATIONS

The present application claims priority under 35 U.S.C. §119 to Japanese Patent Application No. 2015-190861, filed on Sep. 29, 2015. Each of the above application(s) is hereby expressly incorporated by reference, in its entirety, into the present application.

### BACKGROUND OF THE INVENTION

1. Field of the Invention

The present invention relates to a cell evaluation device, method, and a recording medium storing a program for evaluating the degree of progress of differentiation of cells in the process of performing the induction of differentiation of cells.

2. Description of the Related Art

Pluripotent stem cells, such as induced pluripotent stem (iPS) cells or embryonic stem (ES) cells, have the ability to differentiate into cells of various tissues. Accordingly, since pluripotent stem cells can be applied in regenerative medicine, development of medicine, elucidation of disease, and the like, pluripotent stem cells have been drawing attention.

In order to obtain desired cells, such as nerve cells or liver cells, from pluripotent stem cells, induction of differentiation is performed. However, in order to obtain desired cells more efficiently, it is important to check to what extent differentiation has progressed in the process of induction of differentiation.

For example, JP2010-181391A has proposed evaluating the degree of differentiation of cells based on chemical composition analysis of cells using Raman scattering spectra. JP2009-044974A discloses capturing an image of cells at two or more different points in time of a culture period, building a predictive model by analyzing the image at each point in time, and evaluating the degree of differentiation of cells at the present time by comparing the analysis result of the image of cells at the present time with the predictive model.

### SUMMARY OF THE INVENTION

However, the degree of differentiation evaluated in JP2010-181391A or JP2009-044974A is an indicator that simply indicates the degree of differentiation. Accordingly, it is not possible to ascertain the remaining time until desired cell species are obtained, that is, the remaining time until the end of the induction of differentiation, based on only the degree of differentiation. That is, it is not possible to ascertain to what extent differentiation has progressed for the entire process of induction of differentiation from the start of the induction of differentiation to the end of the induction of differentiation.

Specifically, in the induction of differentiation, the degree of the induction of differentiation may differ depending on a culture method or a cell strain, or the progress of differentiation with respect to the passage of time may not be constant. For example, in a case where the degree of differentiation has changed as shown in FIG. 10, the degree of differentiation is high at time t1, but has not yet reached the degree of differentiation at time t2 of the end of the induction of differentiation. Accordingly, even though more time is required until the end of the induction of differentiation, this is likely to be erroneously recognized as the approximate end of the induction of differentiation. Since this affects the scheduling of culturing and the like, it is not possible to obtain desired cells efficiently.

In addition, there is a method of calculating the morphological similarity between cells in the process of induction of differentiation and desired cells and checking the degree of differentiation from the morphological similarity. However, this case can also be said to be the same as the case of calculating the degree of differentiation.

In view of the aforementioned problems, it is an object of the invention to provide a cell evaluation device, method, and a recording medium storing a program capable of checking to what extent differentiation has progressed for the entire process of induction of differentiation from the start of the induction of differentiation to the end of the induction of differentiation.

A cell evaluation device of the invention comprises: a first evaluation unit that evaluates a similarity between cells to be evaluated and cell species, which are located closer to a non-differentiation side than the cells to be evaluated are in a process of differentiation of the cells to be evaluated, as a first similarity; a second evaluation unit that evaluates a similarity between the cells to be evaluated and cell species, which are located closer to a differentiation side than the cells to be evaluated are in the process of differentiation of the cells to be evaluated, as a second similarity; and a differentiation progress calculation unit that calculates a degree of progress of differentiation of the cells to be evaluated based on the first similarity and the second similarity.

In the cell evaluation device of the invention described above, the differentiation progress calculation unit can store a relationship between the first similarity and the second similarity in a process of differentiation of reference cells, which are the same species as the cells to be evaluated, in advance, and calculate the degree of progress of differentiation of the cells to be evaluated based on the relationship between the first similarity and the second similarity stored in advance and the first similarity and the second similarity obtained by evaluating the cells to be evaluated.

In the cell evaluation device of the invention described above, the differentiation progress calculation unit can show the relationship between the first similarity and the second similarity of the reference cells, which is stored in advance, as a straight line or a curve on a two-dimensional coordinate space, find a point on the straight line or the curve corresponding to the first similarity and the second similarity of the cells to be evaluated, and calculate the degree of progress of differentiation of the cells to be evaluated based on lengths of straight lines or curves partitioned off by the point.

In the cell evaluation device of the invention described above, the differentiation progress calculation unit can set a straight line on the two-dimensional coordinate space based on the first similarity and the second similarity of the cells to be evaluated, find an intersection between the straight line and a straight line or a curve showing the relationship between the first similarity and the second similarity stored in advance, divide the straight line or the curve showing the relationship between the first similarity and the second similarity stored in advance by the intersection, and calculate the degree of progress of differentiation of the cells to be evaluated based on lengths of partitioned-off straight lines or curves.

In the cell evaluation device of the invention described above, in a case where there is a plurality of intersections between the straight line based on the first similarity and the second similarity of the cells to be evaluated and the straight line or the curve showing the relationship between the first similarity and the second similarity stored in advance, the differentiation progress calculation unit can select one of the plurality of intersections based on a positional relationship with intersections determined in a previous calculation of the degree of progress of differentiation.

In the cell evaluation device of the invention described above, in a case where there is a plurality of intersections between the straight line based on the first similarity and the second similarity of the cells to be evaluated and the straight line or the curve showing the relationship between the first similarity and the second similarity stored in advance, the differentiation progress calculation unit can select one of the plurality of intersections based on elapsed time information corresponding to a point on the straight line or the curve showing the relationship between the first similarity and the second similarity stored in advance.

In the cell evaluation device of the invention described above, the differentiation progress calculation unit can calculate the degree of progress of differentiation of the cells to be evaluated based on an angle between an axis on the two-dimensional coordinate space and a straight line based on the first similarity and the second similarity of the cells to be evaluated.

In the cell evaluation device of the invention described above, the differentiation progress calculation unit can determine whether or not the first similarity and the second similarity of the cells to be evaluated are included in an allowable range, which is set in advance on the two-dimensional coordinate space based on the straight line or the curve showing the relationship between the first similarity and the second similarity of the reference cells stored in advance, and evaluate the cells to be evaluated as abnormal cells in a case where it is determined that the first similarity and the second similarity of the cells to be evaluated are not included in the allowable range.

The cell evaluation device of the invention described above can further comprise a display control unit that plots a position, which is based on the first similarity and the second similarity of the cells to be evaluated, in the two-dimensional coordinate space to display the position on a display unit.

In the cell evaluation device of the invention described above, the display control unit can plot the straight line or the curve, which shows the relationship between the first similarity and the second similarity of the reference cells stored in advance, in the two-dimensional coordinate space to display the straight line or the curve on the display unit.

A cell evaluation method of the invention comprises: automatically evaluating a similarity between cells to be evaluated and cell species, which are located closer to a non-differentiation side than the cells to be evaluated are in a process of differentiation of the cells to be evaluated, as a first similarity; automatically evaluating a similarity between the cells to be evaluated and cell species, which are located closer to a differentiation side than the cells to be evaluated are in the process of differentiation of the cells to be evaluated, as a second similarity; and automatically calculating a degree of progress of differentiation of the cells to be evaluated based on the first similarity and the second similarity.

A recording medium storing a cell evaluation program of the invention causes a computer to function as: a first evaluation unit that evaluates a similarity between cells to be evaluated and cell species, which are located closer to a non-differentiation side than the cells to be evaluated are in a process of differentiation of the cells to be evaluated, as a first similarity; a second evaluation unit that evaluates a similarity between the cells to be evaluated and cell species, which are located closer to a differentiation side than the cells to be evaluated are in the process of differentiation of the cells to be evaluated, as a second similarity; and a differentiation progress calculation unit that calculates a degree of progress of differentiation of the cells to be evaluated based on the first similarity and the second similarity.

According to the cell evaluation system, method, and recording medium storing a program of the invention, the similarity between the cells to be evaluated and cell species located closer to the non-differentiation side than the cells to be evaluated are is evaluated as the first similarity, the similarity between the cells to be evaluated and cell species located closer to the differentiation side than the cells to be evaluated are is evaluated as the second similarity, and the degree of progress of differentiation of the cells to be evaluated is calculated based on the first similarity and the second similarity. Since the degree of progress of differentiation is an indicator that indicates to what extent differentiation has progressed for the entire process of induction of differentiation from the start of the induction of differentiation to the end of the induction of differentiation, the user can make a schedule for culturing by checking the degree of progress of differentiation. Therefore, it is possible to obtain desired cells more efficiently.

### BRIEF DESCRIPTION OF THE DRAWINGS

FIG. 1 is a block diagram showing the schematic configuration of a cell evaluation system using an embodiment of a cell evaluation device of the invention.

FIG. 2 is a diagram illustrating a method of calculating the degree of progress of differentiation.

FIG. 3 is a flowchart illustrating the operation of the cell evaluation system using an embodiment of the cell evaluation device of the invention.

FIG. 4 is a diagram illustrating a method of determining an abnormal cell.

FIG. 5 is a diagram illustrating a case where there is a plurality of intersections between a curve showing the relationship between the first similarity and the second similarity of reference cells and a straight line based on the first similarity and the second similarity of cells to be evaluated.

FIG. 6 is a diagram illustrating a case where there is a plurality of intersections between a curve showing the relationship between the first similarity and the second similarity of reference cells and a straight line based on the first similarity and the second similarity of cells to be evaluated.

FIG. 7 is a diagram illustrating another method of calculating the degree of progress of differentiation.

FIG. 8 is a diagram illustrating still another method of calculating the degree of progress of differentiation.

FIG. 9 is a diagram illustrating a method of calculating the degree of progress of differentiation from three-dimensional information.

FIG. 10 is a diagram showing an example of a change in the degree of differentiation.

### DESCRIPTION OF THE PREFERRED EMBODIMENTS

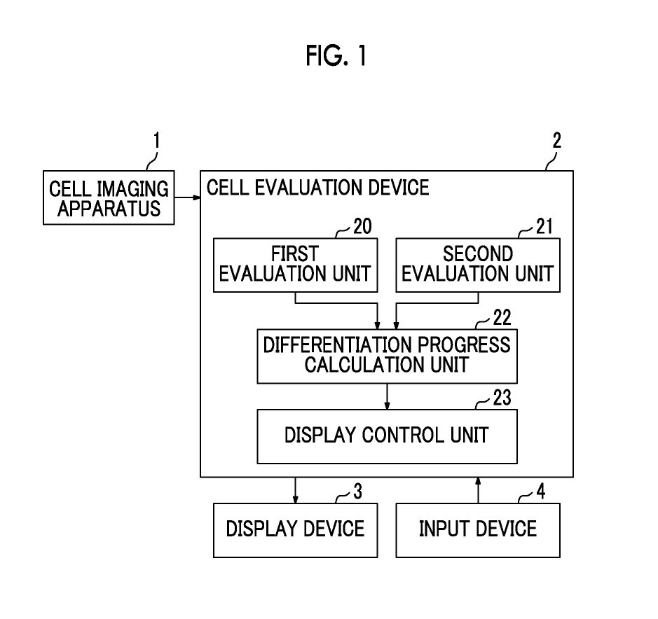

Hereinafter, a cell evaluation system using an embodiment of a cell evaluation device, method, and program of the invention will be described in detail with reference to the diagrams. FIG. 1 is a block diagram showing the schematic configuration of a cell evaluation system using a cell evaluation device of the present embodiment.

As shown in FIG. 1, the cell evaluation system of the present embodiment includes a cell imaging apparatus 1, a cell evaluation device 2, a display device 3, and an input device 4.

The cell imaging apparatus 1 captures an image of cells in the process of induction of differentiation. As the cell imaging apparatus 1, specifically, a phase contrast microscope apparatus, a bright-field microscope apparatus, a differential interference microscope apparatus, a fluorescent observation microscope apparatus, and the like can be used. However, other known microscope apparatuses may also be used. The cell imaging apparatus 1 includes an imaging device, such as a charge-coupled device (CCD) sensor or a complementary metal-oxide semiconductor (CMOS) sensor, and a capture image that has been captured by the imaging device is output to the cell evaluation device 2 from the cell imaging apparatus 1.

As cells to be imaged, for example, there are cells in the process of induction of differentiation from pluripotent stem cells, such as iPS cells and ES cells, to mesoderm, cells in the process of induction of differentiation from neural stem cells to neurons, cells in the process of induction of differentiation from liver stem cells to liver cells, cells in the process of induction of differentiation from pluripotent stem cells to myocardial cells, and cells in the process of induction of differentiation from hematopoietic stem cells to red blood cells, lymphocytes, or platelets. However, cells to be imaged are not limited thereto, and may be cells in the other processes of induction of differentiation.

The cell evaluation device 2 calculates the degree of progress of differentiation of cells included in a captured image based on the input captured image. The degree of progress of differentiation shows to what extent differentiation has progressed for the entire process of induction of differentiation from the start of the induction of differentiation to a time when desired cell species are obtained, that is, to the end of the induction of differentiation.

Specifically, the cell evaluation device 2 calculates the similarity between cells to be evaluated and two cell species, and calculates the degree of progress of differentiation of cells to be evaluated based on the similarity between the cells to be evaluated and the two cell species. The two cell species are cell species located closer to the non-differentiation side than the cells to be evaluated are and cell species located closer to the differentiation side than the cells to be evaluated are in the process of differentiation of the cells to be evaluated.

Specifically, as shown in FIG. 1, the cell evaluation device 2 includes a first evaluation unit 20, a second evaluation unit 21, a differentiation progress calculation unit 22, and a display control unit 23.

The cell evaluation device 2 is a computer configured to include a central processing unit (CPU), a semiconductor memory, and a storage device such as a hard disk or a solid state drive (SSD). A cell evaluation program of the present embodiment is installed in the storage device, and the central processing unit executes the cell evaluation program to operate the first evaluation unit 20, the second evaluation unit 21, the differentiation progress calculation unit 22, and the display control unit 23 described above.

The first evaluation unit 20 evaluates the similarity between the cells to be evaluated and cell species, which are located closer to the non-differentiation side than the cells to be evaluated are in the process of differentiation of the cells to be evaluated, as a first similarity.

For example, in the case of calculating the degree of progress of differentiation of cells in the process of differentiation from pluripotent stem cells to mesoderm, the similarity between the cells to be evaluated and the pluripotent stem cells is calculated as the first similarity. In the case of calculating the degree of progress of differentiation of cells in the process of differentiation from neural stem cells to neurons, the similarity between the cells to be evaluated and the neural stem cells is calculated as the first similarity.

In the case of calculating the degree of progress of differentiation of cells in the process of differentiation from liver stem cells to liver cells, the similarity between the cells to be evaluated and the liver stem cells is calculated as the first similarity. In the case of calculating the degree of progress of differentiation of cells in the process of differentiation from pluripotent stem cells to myocardial cells, the similarity between the cells to be evaluated and the pluripotent stem cells is calculated as the first similarity.

The first similarity is the morphological similarity between the cells to be evaluated and cell species on the non-differentiation side. The first evaluation unit 20 includes a discriminator of cell species on the non-differentiation side generated using, for example, a method of machine learning, and calculates the first similarity by inputting an image of the cells to be evaluated to the discriminator. A known technique can be used for the machine learning, and methods, such as a neural network, a support vector machine, and Adaboost, can be mentioned. For a method of calculating the morphological similarity, it is possible to use a known technique.

The discriminator is generated by machine learning in the feature quantity space based on various feature quantities. At this time, in the feature quantity space for learning, multi-dimensional feature quantities, such as a co-occurrence matrix is used. By the machine learning, it is possible to find a valid identification surface.

The second evaluation unit 21 evaluates the similarity between the cells to be evaluated and cell species, which are located closer to the differentiation side than the cells to be evaluated are in the process of differentiation of the cells to be evaluated, as a second similarity.

For example, in the case of calculating the degree of progress of differentiation of cells in the process of differentiation from pluripotent stem cells to mesoderm, the similarity between the cells to be evaluated and the mesoderm is calculated as the second similarity. In addition, in the case of calculating the degree of progress of differentiation of cells in the process of differentiation from neural stem cells to neurons, the similarity between the cells to be evaluated and the neurons is calculated as the second similarity.

In the case of calculating the degree of progress of differentiation of cells in the process of differentiation from liver stem cells to liver cells, the similarity between the cells to be evaluated and the liver cells is calculated as the second similarity. In the case of calculating the degree of progress of differentiation of cells in the process of differentiation from pluripotent stem cells to, the similarity between the cells to be evaluated and the myocardial cells is calculated as the second similarity.

The second similarity is the morphological similarity between the cells to be evaluated and cell species on the differentiation side. Similarly to the first evaluation unit 20, the second evaluation unit 21 includes a discriminator of cell species on the differentiation side generated using, for example, a method of machine learning, and calculates the second similarity by inputting an image of the cells to be evaluated to the discriminator.

The differentiation progress calculation unit 22 calculates the degree of progress of differentiation of the cells to be evaluated based on the first similarity calculated by the first evaluation unit 20 and the second similarity calculated by the second evaluation unit 21. Hereinafter, a method of calculating the degree of progress of differentiation of the present embodiment will be described.

The differentiation progress calculation unit 22 stores the relationship between the first similarity and the second similarity in the process of differentiation of cells as a reference (cells that normally differentiate to become a reference; hereinafter, referred to as reference cells), which are the same species as the cells to be evaluated, in advance. Then, based on the relationship between the first similarity and the second similarity stored in advance and the first similarity and the second similarity obtained by evaluating the cells to be evaluated, the degree of progress of differentiation of the cells to be evaluated is calculated.

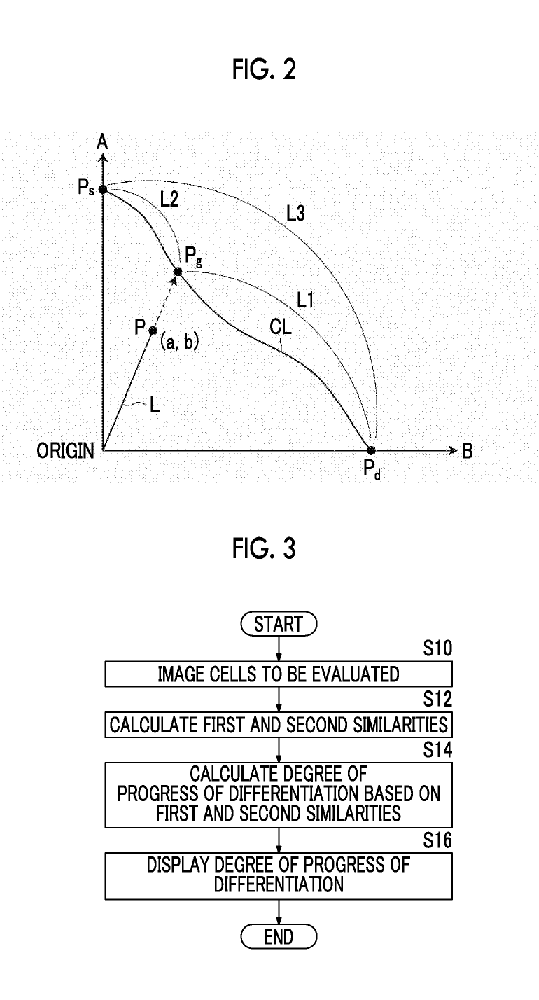

Specifically, the differentiation progress calculation unit 22 shows the relationship between the first similarity and the second similarity of the reference cells as a straight line or a curve on the two-dimensional coordinate space. FIG. 2 is an example showing the relationship between the first similarity and the second similarity stored in advance as a curve CL. However, the relationship between the first similarity and the second similarity may be expressed in a straight line as described above.

In the two-dimensional coordinate space shown in FIG. 2, a vertical axis A is an axis showing the first similarity, and a horizontal axis B is an axis showing the second similarity. In the present embodiment, a point indicating the first similarity and the second similarity at the start of induction of differentiation of reference cells is set as a point Ps on the vertical axis A, and a point indicating the first similarity and the second similarity at the end of induction of differentiation of reference cells is set as a point Pd on the horizontal axis B.

The differentiation progress calculation unit 22 finds a point on the curve corresponding to the first similarity and the second similarity of the cells to be evaluated, and calculates the degree of progress of differentiation of the cells to be evaluated based on the lengths of curves partitioned off by the point. Specifically, as shown in FIG. 2, the differentiation progress calculation unit 22 sets a straight line L on the two-dimensional coordinate space by connecting a point P, which is expressed by the first similarity a and the second similarity b of the cells to be evaluated, and the origin to each other. Then, the differentiation progress calculation unit 22 finds an intersection Pg between the straight line L and the curve CL, and divides the curve CL by the intersection Pg, and calculates the degree of progress of differentiation of the cells to be evaluated based on the lengths of curves CL partitioned off by the intersection Pg.

For example, in a case where the length of a line between the intersection Pg and the point Pd is set to L1 and the length of a line between the point Ps and the point Pd is set to L3, the degree of progress of differentiation is calculated by L1/L3. In a case where the length of a line between the point Ps and the intersection Pg is set to L2, L2/L3 may be calculated as the degree of progress of differentiation.

The display control unit 23 displays the captured image of the cells to be evaluated, which is output from the cell imaging apparatus 1, on the display device 3. In addition, the display control unit 23 displays the degree of progress of differentiation of the cells to be evaluated, which is calculated by the differentiation progress calculation unit 22, on the display device 3.

In addition to displaying the degree of progress of differentiation, the display control unit 23 may display the coordinate axes shown in FIG. 2 on the display device 3 to plot the curve CL showing the relationship between the first similarity and the second similarity of the reference cells or a position (point), which is based on the first similarity and the second similarity of the cells to be evaluated, on the coordinate axes.

The display device 3 (corresponding to a display unit of the invention) is formed by a liquid crystal display or the like. The input device 4 is formed by a keyboard, a mouse, or the like. The display device 3 may also be used as the input device 4 by forming the display device 3 and the input device 4 using a touch panel.

Next, the operation of the cell evaluation system of the present embodiment will be described with reference to the flowchart shown in FIG. 3.

First, cells to be evaluated in the process of induction of differentiation are placed in the cell imaging apparatus 1, and an image of the cells to be evaluated is captured (S10).

The image of cells captured by the cell imaging apparatus 1 is input to the first evaluation unit 20 and the second evaluation unit 21 of the cell evaluation device 2. Then, the first similarity is calculated by the first evaluation unit 20, and the second similarity is calculated by the second evaluation unit 21 (S12).

Then, the first and second similarities calculated for the cells to be evaluated are input to the differentiation progress calculation unit 22, and the differentiation progress calculation unit 22 calculates the degree of progress of differentiation of the cells to be evaluated based on the input first and second similarities (S14). Specifically, a curve showing the relationship between the first similarity and the second similarity of the reference cells is set on the two-dimensional coordinate space in which the vertical axis is set as an axis indicating the first similarity and the horizontal axis is set as an axis indicating the second similarity as described above.

Then, the differentiation progress calculation unit 22 sets a straight line on the two-dimensional coordinate space by connecting a point, which is expressed by the first similarity and the second similarity of the cells to be evaluated, and the origin to each other, and finds an intersection between the straight line and the curve. Then, the differentiation progress calculation unit 22 divides the curve by the intersection, and calculates the degree of progress of differentiation of the cells to be evaluated based on the lengths of partitioned-off curves.

The degree of progress of differentiation calculated by the differentiation progress calculation unit 22 is output to the display control unit 23, and the display control unit 23 displays the input degree of progress of differentiation on the display device 3 (S16).

According to the cell evaluation system of the embodiment described above, the similarity between the cells to be evaluated and cell species located closer to the non-differentiation side than the cells to be evaluated are is evaluated as the first similarity, the similarity between the cells to be evaluated and cell species located closer to the differentiation side than the cells to be evaluated are is evaluated as the second similarity, and the degree of progress of differentiation of the cells to be evaluated is calculated based on the first similarity and the second similarity. Since the user can make a schedule of culturing by checking the degree of progress of differentiation, it is possible to obtain desired cells more efficiently.

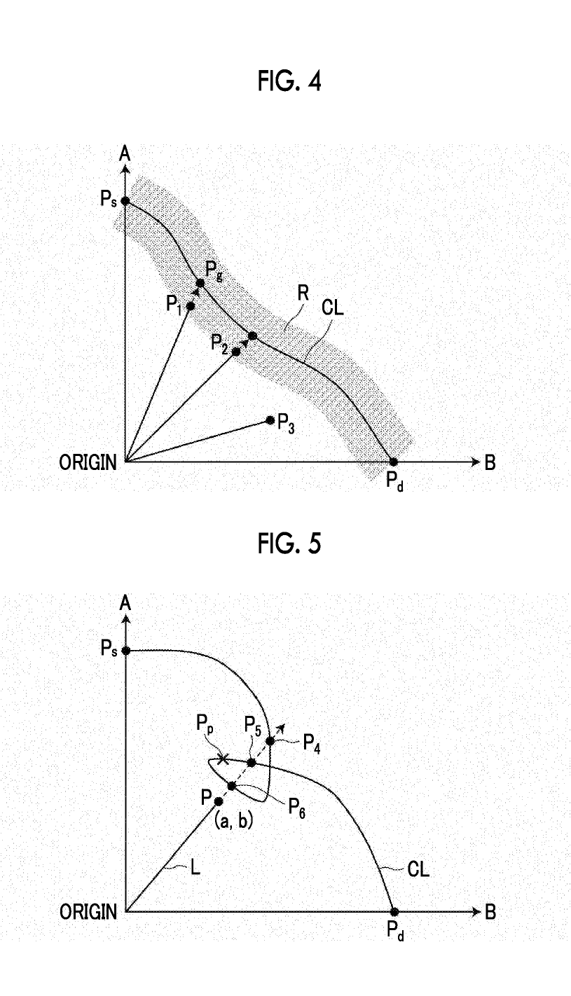

In the cell evaluation system of the embodiment described above, in a case where the differentiation progress calculation unit 22 calculates the degree of progress of differentiation of the cells to be evaluated, an allowable range R for which it can be regarded that differentiation has normally progressed may be set on the two-dimensional coordinate space based on the curve CL showing the relationship between the first similarity and the second similarity of the reference cells, and it may be determined whether or not a point expressed by the first similarity and the second similarity of the cells to be evaluated is included in the allowable range R, as shown in FIG. 4.

Then, in a case where it is determined that the point expressed by the first similarity and the second similarity of the cells to be evaluated is not included in the allowable range R, the cells to be evaluated may be evaluated as abnormal cells, and information indicating that the point expressed by the first similarity and the second similarity of the cells to be evaluated is not included in the allowable range R may be displayed on the display device 3 so that the user is notified of the determination result. In the case of the example shown in FIG. 4, in a case where the points expressed by the first similarity and the second similarity of the cells to be evaluated are a point P₁ and a point P₂, the cells to be evaluated are evaluated as normal cells and the degree of progress of differentiation is evaluated. However, in a case where the point expressed by the first similarity and the second similarity of the cells to be evaluated is a point P₃, the cells to be evaluated are evaluated as abnormal cells, and the degree of progress of differentiation is not evaluated and the user is notified of the evaluation result indicating that the cells to be evaluated are evaluated as abnormal cells.

In the embodiment described above, in a case where the differentiation progress calculation unit 22 calculates the degree of progress of differentiation of the cells to be evaluated, in a case where the curve CL showing the relationship between the first similarity and the second similarity of the reference cells is a shape shown in FIG. 5, a plurality of intersections between the straight line L and the curve CL based on the first similarity and the second similarity of the cells to be evaluated are present.

Thus, in a case where a plurality of intersections are present, the differentiation progress calculation unit 22 may select one of the plurality of intersections based on the positional relationship with intersections determined in the previous calculation of the degree of progress of differentiation. For example, as shown in FIG. 5, in a case where intersections P₄ to P₆ are present, the intersection P₅ is selected in a case where the intersection determined in the previous calculation of the degree of progress of differentiation is Pₚ since time passes in a direction from the point Ps to the point Pd in the curve CL.

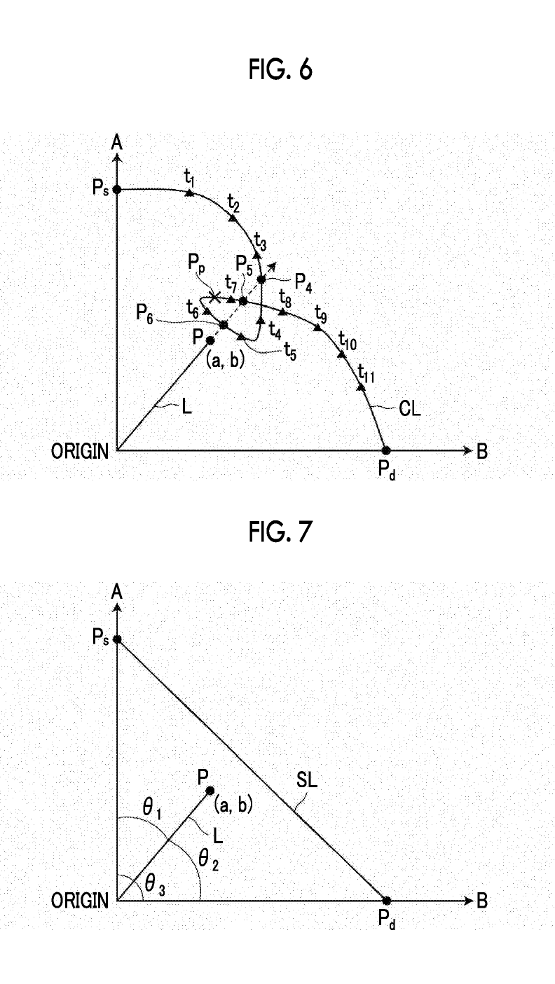

The intersection selection method is not limited to the above method. For example, as shown in FIG. 6, elapsed time information t₁ to t₁₁ from the start of the induction of differentiation may be added and stored for each point on the curve CL showing the relationship between the first similarity and the second similarity of the reference cells, elapsed time information closest to the elapsed time from the start of the induction of differentiation at the present calculation time of the degree of progress of differentiation may be searched for among the pieces of elapsed time information t₁ to t₁₁, and an intersection closest to the point to which the elapsed time information has been added may be selected. For example, in a case where the elapsed time information closest to the elapsed time from the start of the induction of differentiation at the calculation time of the degree of progress of differentiation is t₇, an intersection P₅ closest to the point to which the elapsed time information t₇ has been added is selected.

In the embodiment described above, the degree of progress of differentiation is calculated based on the intersections between the straight line connecting the point P, which is expressed by the first similarity and the second similarity of the cells to be evaluated, and the origin to each other and the curve CL showing the relationship between the first similarity and the second similarity of the reference cells. However, the method of calculating the degree of progress of differentiation is not limited thereto. For example, in a case where the relationship between the first similarity and the second similarity of the reference cells is expressed by a straight line SL or expressed by a curve close to the straight line as shown in FIG. 7, the degree of progress of differentiation may be calculated based on the angle of the straight line L, which is based on the first similarity and the second similarity of the cells to be evaluated, on the two-dimensional coordinate space.

Specifically, as shown in FIG. 7, in a case where the angle between the straight line L connecting the point P and the origin to each other and the vertical axis (straight line connecting the point Ps and the origin to each other) is θ₁ and the angle between the vertical axis and the horizontal axis (straight line connecting the point Pd and the origin to each other) is θ₃, θ₁/θ₃ may be calculated as the degree of progress of differentiation. In addition, in a case where the angle between the straight line L connecting the point P and the origin to each other and the horizontal axis is θ₂, θ₂/θ₃ may be calculated as the degree of progress of differentiation.

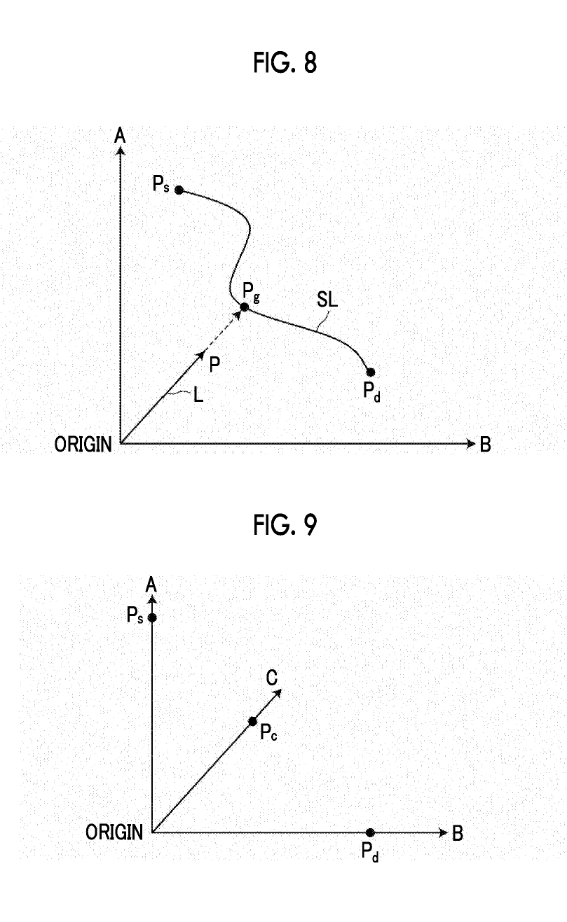

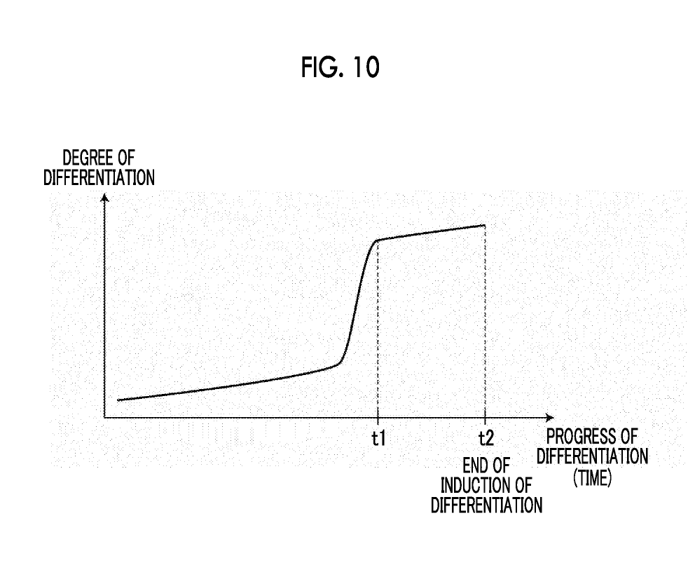

In the embodiment described above, a point indicating the first similarity and the second similarity at the start of induction of differentiation of the reference cells is set on the vertical axis A, and a point indicating the first similarity and the second similarity at the end of induction of differentiation of the reference cells is set on the horizontal axis B. However, without being limited thereto, the point indicating the first similarity and the second similarity at the start of induction of differentiation of the reference cells may be set as a point Ps outside the vertical axis A, and the point indicating the first similarity and the second similarity at the end of induction of differentiation of the reference cells may be set as a point Pd outside the horizontal axis B, as shown in FIG. 8.

In the embodiment described above, the degree of progress of differentiation is calculated based on the two-dimensional information of the first similarity and the second similarity. However, the amount of information used to calculate the degree of progress of differentiation may be further increased to calculate the degree of progress of differentiation based on the three-dimensional information.

Specifically, as shown in FIG. 9, an A axis indicating the first similarity, a B axis indicating the second similarity, and a C axis perpendicular to the A axis and the B axis may be set, and the C axis may be set as an axis indicating the third information.

For example, in the case of calculating the degree of progress of differentiation of cells in the process of differentiation from neural stem cells to neurons, the size, circularity, or brightness of the cells to be evaluated can be used as the third information. In addition, it is also possible to use the length or the number of axons of the cells to be evaluated, the size or the number of dendrites, the size or the number of cell nuclei, the area or density of white streaks indicating the boundary of cells, and the like.

In the case of calculating the degree of progress of differentiation of cells in the process of differentiation from liver stem cells to liver cells, the size, circularity, or brightness of the cells to be evaluated, the size or the number of cell nuclei, the area or density of white streaks described above, and the like can be used as the third information.

In the case of calculating the degree of progress of differentiation of cells in the process of differentiation from pluripotent stem cells to myocardial cells, pulsation of the cells to be evaluated, the area or density of white streaks described above, and the like can be used as the third information. For the information of the pulsation of cells, for example, electrodes may be provided and the electric potential of the cells may be obtained by measurement, or the amount of variation between two images captured in time series may be obtained.

Even in the case of calculating the degree of progress of differentiation based on the three-dimensional information, similarly to the method of calculating the degree of progress of differentiation based on the two-dimensional information, a curve showing the relationship among the first similarity, the second similarity, and the third information of the reference cells may be stored in advance, and the degree of progress of differentiation may be calculated based on the curve. Specifically, for example, an intersection between the curve described above and the straight line based on the first similarity, the second similarity, and the third information of the cells to be evaluated may be found, and the degree of progress of differentiation of the cells to be evaluated may be calculated based on the lengths of curves partitioned off by the intersection.

However, in the case of calculating the degree of progress of differentiation based on the three-dimensional information, there may be no intersection between the curve acquired based on the reference cells and the straight line acquired based on the cells to be evaluated. Therefore, for the curve acquired based on the reference cells, distribution obtained by adding a margin to some extent is desirable. In a case where there is no intersection between the curve acquired based on the reference cells and the straight line acquired based on the cells to be evaluated, the degree of progress of differentiation may be calculated based on the two-dimensional information between the first similarity and the second similarity.

1. A cell evaluation device, comprising:
a first evaluation unit that evaluates a similarity between cells to be evaluated and cell species, which are located closer to a non-differentiation side than the cells to be evaluated are in a process of differentiation of the cells to be evaluated, as a first similarity;
a second evaluation unit that evaluates a similarity between the cells to be evaluated and cell species, which are located closer to a differentiation side than the cells to be evaluated are in the process of differentiation of the cells to be evaluated, as a second similarity; and
a differentiation progress calculation unit that calculates a degree of progress of differentiation of the cells to be evaluated based on the first similarity and the second similarity.

2. The cell evaluation device according to claim 1,
wherein the differentiation progress calculation unit stores a relationship between the first similarity and the second similarity in a process of differentiation of reference cells, which are the same species as the cells to be evaluated, in advance, and calculates the degree of progress of differentiation of the cells to be evaluated based on the relationship between the first similarity and the second similarity stored in advance and the first similarity and the second similarity obtained by evaluating the cells to be evaluated.

3. The cell evaluation device according to claim 2,
wherein the differentiation progress calculation unit shows the relationship between the first similarity and the second similarity of the reference cells, which is stored in advance, as a straight line or a curve on a two-dimensional coordinate space, finds a point on the straight line or the curve corresponding to the first similarity and the second similarity of the cells to be evaluated, and calculates the degree of progress of differentiation of the cells to be evaluated based on lengths of the straight lines or the curves partitioned off by the point.

4. The cell evaluation device according to claim 3,
wherein the differentiation progress calculation unit sets a straight line on the two-dimensional coordinate space based on the first similarity and the second similarity of the cells to be evaluated, finds an intersection between the straight line and a straight line or a curve showing the relationship between the first similarity and the second similarity stored in advance, divides the straight line or the curve showing the relationship between the first similarity and the second similarity stored in advance by the intersection, and calculates the degree of progress of differentiation of the cells to be evaluated based on lengths of partitioned-off straight lines or curves.

5. The cell evaluation device according to claim 4,
wherein, in a case where there is a plurality of intersections between the straight line based on the first similarity and the second similarity of the cells to be evaluated and the straight line or the curve showing the relationship between the first similarity and the second similarity stored in advance, the differentiation progress calculation unit selects one of the plurality of intersections based on a positional relationship with intersections determined in a previous calculation of the degree of progress of differentiation.

6. The cell evaluation device according to claim 4,
wherein, in a case where there is a plurality of intersections between the straight line based on the first similarity and the second similarity of the cells to be evaluated and the straight line or the curve showing the relationship between the first similarity and the second similarity stored in advance, the differentiation progress calculation unit selects one of the plurality of intersections based on elapsed time information corresponding to a point on the straight line or the curve showing the relationship between the first similarity and the second similarity stored in advance.

7. The cell evaluation device according to claim 3,
wherein the differentiation progress calculation unit calculates the degree of progress of differentiation of the cells to be evaluated based on an angle between an axis on the two-dimensional coordinate space and a straight line based on the first similarity and the second similarity of the cells to be evaluated.

8. The cell evaluation device according to claim 3,
wherein the differentiation progress calculation unit determines whether or not the first similarity and the second similarity of the cells to be evaluated are included in an allowable range, which is set in advance on the two-dimensional coordinate space based on the straight line or the curve showing the relationship between the first similarity and the second similarity of the reference cells stored in advance, and evaluates the cells to be evaluated as abnormal cells in a case where it is determined that the first similarity and the second similarity of the cells to be evaluated are not included in the allowable range.

9. The cell evaluation device according to claim 4,
wherein the differentiation progress calculation unit determines whether or not the first similarity and the second similarity of the cells to be evaluated are included in an allowable range, which is set in advance on the two-dimensional coordinate space based on the straight line or the curve showing the relationship between the first similarity and the second similarity of the reference cells stored in advance, and evaluates the cells to be evaluated as abnormal cells in a case where it is determined that the first similarity and the second similarity of the cells to be evaluated are not included in the allowable range.

10. The cell evaluation device according to claim 5,
wherein the differentiation progress calculation unit determines whether or not the first similarity and the second similarity of the cells to be evaluated are included in an allowable range, which is set in advance on the two-dimensional coordinate space based on the straight line or the curve showing the relationship between the first similarity and the second similarity of the reference cells stored in advance, and evaluates the cells to be evaluated as abnormal cells in a case where it is determined that the first similarity and the second similarity of the cells to be evaluated are not included in the allowable range.

11. The cell evaluation device according to claim 6,
wherein the differentiation progress calculation unit determines whether or not the first similarity and the second similarity of the cells to be evaluated are included in an allowable range, which is set in advance on the two-dimensional coordinate space based on the straight line or the curve showing the relationship between the first similarity and the second similarity of the reference cells stored in advance, and evaluates the cells to be evaluated as abnormal cells in a case where it is determined that the first similarity and the second similarity of the cells to be evaluated are not included in the allowable range.

12. The cell evaluation device according to claim 3, further comprising:
a display control unit that plots a position, which is based on the first similarity and the second similarity of the cells to be evaluated, in the two-dimensional coordinate space to display the position on a display unit.

13. The cell evaluation device according to claim 4, further comprising:
a display control unit that plots a position, which is based on the first similarity and the second similarity of the cells to be evaluated, in the two-dimensional coordinate space to display the position on a display unit.

14. The cell evaluation device according to claim 5, further comprising:
a display control unit that plots a position, which is based on the first similarity and the second similarity of the cells to be evaluated, in the two-dimensional coordinate space to display the position on a display unit.

15. The cell evaluation device according to claim 6, further comprising:
a display control unit that plots a position, which is based on the first similarity and the second similarity of the cells to be evaluated, in the two-dimensional coordinate space to display the position on a display unit.

16. The cell evaluation device according to claim 9,
wherein the display control unit plots the straight line or the curve, which shows the relationship between the first similarity and the second similarity of the reference cells stored in advance, in the two-dimensional coordinate space to display the straight line or the curve on the display unit.

17. The cell evaluation device according to claim 10,
wherein the display control unit plots the straight line or the curve, which shows the relationship between the first similarity and the second similarity of the reference cells stored in advance, in the two-dimensional coordinate space to display the straight line or the curve on the display unit.

18. The cell evaluation device according to claim 11,
wherein the display control unit plots the straight line or the curve, which shows the relationship between the first similarity and the second similarity of the reference cells stored in advance, in the two-dimensional coordinate space to display the straight line or the curve on the display unit.

19. A cell evaluation method using the cell evaluation device according to claim 1, comprising:
automatically evaluating a similarity between cells to be evaluated and cell species, which are located closer to a non-differentiation side than the cells to be evaluated are in a process of differentiation of the cells to be evaluated, as a first similarity;
automatically evaluating a similarity between the cells to be evaluated and cell species, which are located closer to a differentiation side than the cells to be evaluated are in the process of differentiation of the cells to be evaluated, as a second similarity; and
automatically calculating a degree of progress of differentiation of the cells to be evaluated based on the first similarity and the second similarity.

20. A non-transitory recording medium storing a cell evaluation program to execute the cell evaluation method according to claim 19, causing a computer to function as:
a first evaluation unit that evaluates a similarity between cells to be evaluated and cell species, which are located closer to a non-differentiation side than the cells to be evaluated are in a process of differentiation of the cells to be evaluated, as a first similarity;
a second evaluation unit that evaluates a similarity between the cells to be evaluated and cell species, which are located closer to a differentiation side than the cells to be evaluated are in the process of differentiation of the cells to be evaluated, as a second similarity; and
a differentiation progress calculation unit that calculates a degree of progress of differentiation of the cells to be evaluated based on the first similarity and the second similarity.
